(19) United States
(12) Patent Application Publication
(10) Pub. No.: US 2022/0125763 A1
(43) Pub. Date: Apr. 28, 2022

(54) COMPOSITIONS PROVIDING ENHANCED ANTIBACTERIAL ACTIVITY AGAINST GRAM-POSITIVE BACTERIA AND USE THEREOF

(71) Applicant: ANIFERA LIMITED, London (GB)

(72) Inventors: David JUNG, Durham, NC (US); Daina ZENG, Cary, NC (US)

(21) Appl. No.: 17/432,279

(22) PCT Filed: Feb. 19, 2020
(86) PCT No.: PCT/US2020/018724

§ 371 Date: Aug. 19, 2021

Related U.S. Application Data
(60) Provisional application No. 62/808,900, filed on Feb. 22, 2019.

(51) Int. Cl. A61K 31/4168; A61K 31/43; A61K 38/12; A61K 38/14; A61K 31/431; A61K 31/5377; A61P 31/04

(52) U.S. Cl. CPC A61K 31/4168; A61K 31/43; A61K 38/12; A61P 31/04; A61K 31/431; A61K 31/5377; A61K 38/14

(57) ABSTRACT

A method of inhibiting, reducing growth of or destroying gram positive bacteria comprising contacting the gram positive bacteria with an effective amount of a 2-(substituted-amino)-imidazole compound and with an additional antibacterial compound separately, simultaneously, or sequentially, whereby the two compounds provide an antibiotic potentiation effect against the gram-positive bacteria. The additional antibacterial compound may comprise penicillin, daptomycin, vancomycin, oxacillin, linezolid, or a related antibiotic(s).

### CROSS REFERENCE TO RELATED APPLICATIONS

This application claims the benefit of U.S. provisional patent application No. 62/808,900 filed Feb. 22, 2019, the disclosure of which is hereby incorporated by reference in its entirety.

### TECHNICAL FIELD

Described herein are antibacterial compositions that provide enhanced antibacterial activity against gram positive bacteria. The antibacterial compositions may be used in combination with additional antibiotic compounds and/or compositions against gram positive bacteria to provide an antibiotic potentiation effect. In embodiments, the antibacterial compositions include 2-(substituted-amino)-imidazole compounds. The antibacterial compositions may be used in human and animal health applications to inhibit or reduce growth of and/or destroy gram positive bacteria.

### BACKGROUND

The development of drug resistant bacteria is a major problem in medicine as more antibiotics are used for a wide variety of illnesses and other conditions. The use of more antibiotics and the number of bacteria showing resistance has prompted longer treatment times. Furthermore, broad, non-specific antibiotics, some of which have detrimental effects on subjects, are being used more frequently.

Gram-positive bacteria, such as Staphylococci, Enterococci and Clostridia, are important pathogens in both human and veterinary medicine. Gram-positive bacteria include but are not limited to the genera Actinomyces, Bacillus, Listeria, Lactococcus, Staphylococcus, Streptococcus, Enterococcus, Mycobacterium, Corynebacterium, and Clostridium. Medically relevant species include Streptococcus pyogenes, Streptococcus pneumoniae, Staphylococcus aureus, and Enterococcus faecalis. Bacillus species, which are spore-forming, cause anthrax and gastroenteritis. Spore-forming Clostridium species are responsible for botulism, tetanus, gas gangrene and pseudomembranous colitis. Corynebacterium species cause diphtheria, and Listeria species cause meningitis.

Antibiotic resistance in bacteria has arisen through the prolific use of antibiotic drugs both in human medicine and animal husbandry, indiscriminate prescribing practices, and patient non-compliance with treatment regimes. Therapeutic options for the treatment of drug-resistant microorganisms, especially gram-positive bacteria, are becoming increasingly limited. The problem of antibiotic resistance is exacerbated by the spread of drug-resistant organisms, and the dissemination of resistance genes between bacteria. The threat to the successful management of bacterial infections posed by the development and spread of antibiotic resistance is a significant problem within healthcare and veterinary medicine.

Staphylococci are major causes of serious healthcare associated infection (HAI). Of particular note are strains of Staphylococcus that have developed or obtained varying levels of resistance to antibiotics such as methicillin (meticillin). These difficult-to-treat organisms are commonly known as methicillin resistant Staphylococcus aureus (MRSA) and methicillin resistant Staphylococcus epidermidis (MRSE). Approximately 80% of S. epidermidis isolates from device-associated infections are methicillin resistant (MRSE) as well as being multi-resistant.

The broad spectrum antibiotics in clinical use for treatment of gram positive infections are limited in use and application by the development of resistance, particularly in connection with continued or long-term use.

It is evident from the deficiencies and problems associated with current traditional antibacterial agents that there still exists a need in the art for additional specific bacterial agents, combinations and therapeutic modalities, particularly without high risks of acquired resistance. Accordingly, there is a commercial need for new antibacterial approaches, especially those that operate via new modalities or provide new combinations to effectively kill pathogenic bacteria.

The citation of references herein shall not be construed as an admission that such is prior art to the present invention.

### SUMMARY

A first aspect of the invention includes a therapeutically active 2-(substituted-amino)-imidazole compound of the general formula (Formula I), a salt, enantiomer or derivative thereof:

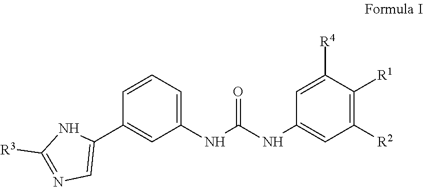

wherein,
R¹, R², and R⁴, which may be the same or different, are each selected from the group consisting of hydrogen, lower alkyl, halogen, and haloalkyl, and
R³ is lower alkylamino, lower isoalkylamino or benzamide.

In a feature of the first aspect, the 2-(substituted-amino)-imidazole compound is selected from the group consisting of the following compounds (Compounds 1-5):

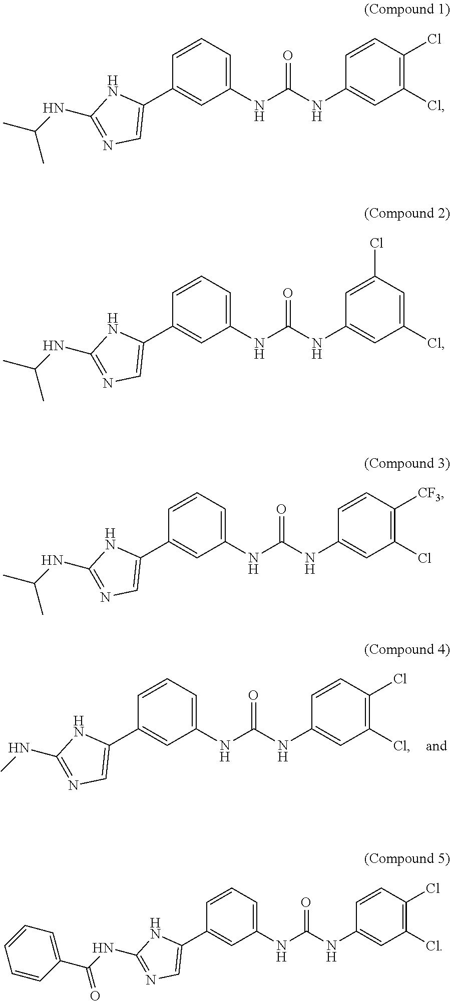 [SMILES: CC(C)Nc1ncc(-c2cccc(NC(=O)Nc3ccc(Cl)c(Cl)c3)c2)[nH]1 ; CC(C)Nc1ncc(-c2cccc(NC(=O)Nc3cc(Cl)cc(Cl)c3)c2)[nH]1 ; CC(C)Nc1ncc(-c2cccc(NC(=O)Nc3ccc(C(F)(F)F)c(Cl)c3)c2)[nH]1 ; CNc1ncc(-c2cccc(NC(=O)Nc3ccc(Cl)c(Cl)c3)c2)[nH]1 ; O=C(Nc1cccc(-c2cnc(NC(=O)c3ccccc3)[nH]2)c1)Nc1ccc(Cl)c(Cl)c1]

In a second aspect of the invention, a method of inhibiting, reducing growth of or destroying gram-positive bacteria includes contacting the gram-positive bacteria with an effective amount of a 2-(substituted-amino)-imidazole compound of the general formula (Formula I), a salt, enantiomer or derivative thereof, and with an additional antibacterial compound separately, simultaneously, or sequentially:

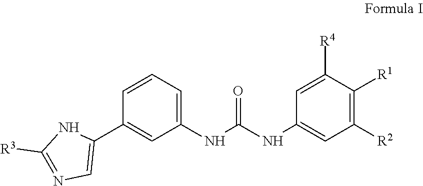

wherein,
R¹, R², and R⁴, which may be the same or different, are each selected from the group consisting of hydrogen, lower alkyl, halogen, and haloalkyl, and
R³ is lower alkylamino, lower isoalkylamino or benzamide.

In a feature of the second aspect, the imidazole compound can be selected from one or more of Compounds 1-5.

In a third aspect of the invention, a method of enhancing antibacterial activity of a first antibacterial compound against gram positive bacteria includes contacting the gram positive bacteria with an effective amount the first antibacterial compound and an effective amount of a 2-(substituted-amino)-imidazole antibacterial compound, a salt, enantiomer or derivative thereof, separately, simultaneously, or sequentially.

In a feature of the third aspect, the imidazole compound can be selected from one or more of Compounds 1-5.

In a feature of the second or third aspect, the first antibacterial compound (or additional antibacterial compound) comprises penicillin, daptomycin, vancomycin, oxacillin, linezolid, or related antibiotic(s). With regard to the second and third aspects,

the gram-positive bacteria comprise one or more of the genera Actinomyces, Bacillus, Listeria, Lactococcus, Staphylococcus, Streptococcus, Enterococcus, Mycobacterium, Corynebacterium, or Clostridium.

In a feature of the third aspect, the minimum inhibitory concentration (MIC) of the first antibacterial compound is reduced by at least 10× when the first antibacterial compound is applied in combination with the 2-(substituted-amino)-imidazole antibacterial compound, a salt, enantiomer or derivative thereof, wherein applied in combination includes application to the gram positive bacteria separately, simultaneously, or sequentially. In additional features, the MIC of the first antibacterial compound is reduced by at least 15× or reduced by at least 25×.

In a fourth aspect of the invention, a method of treating a subject suffering from an infection contributed to or caused by gram-positive bacteria, includes administering an effective amount of a therapeutically active 2-(substituted-amino)-imidazole compound, an enantiomer or salt thereof, and an additional antibacterial compound, separately, simultaneously, or sequentially. The gram-positive bacteria may include one or more of the genera Actinomyces, Bacillus, Listeria, Lactococcus, Staphylococcus, Streptococcus, Enterococcus, Mycobacterium, Corynebacterium, or Clostridium. The subject may be human or animal. The therapeutically active 2-(substituted-amino)-imidazole compound and the antibacterial compound may be administered orally, topically to the site of infection, intravenously, transmucosally, or transdermally. The infection may be bovine mastitis.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a film” can include a plurality of such films, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

Described herein are imidazole compounds that effectively enhance antibiotic activity against gram positive bacteria when combined with other antibiotic compounds (also referred to herein as antibiotics and antibiotic agents). In particular, a group of five 2-(substituted-amino)-imidazole compounds, described below as Compounds 1-5, have shown a potentiation effect for antibiotic activity against gram-positive bacteria.

The term “potentiation” means an interaction between two or more drugs or agents resulting in a pharmacologic response that is greater than the sum of the individual responses for each drug or agent. When a compound has a potentiation effect on another active or drug, the potentiating compound has the effect of making the active or drug more effective or more active than it is alone. The potentiating compound augments, improves, or enhances the activity of the drug or active.

The therapeutically active 2-(substituted-amino)-imidazole compounds have a structure represented by the general formula (Formula I). The imidazole compounds described herein include a salt, enantiomer or derivative of the compound. The general formula is represented as follows:

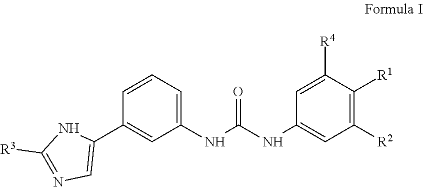

wherein,

R¹, R², and R⁴, which may be the same or different, are each selected from the group consisting of hydrogen, lower alkyl, halogen, and haloalkyl, and

R³ is lower alkylamino, lower isoalkylamino or benzamide.

Exemplary embodiments of the 2-(substituted-amino)-imidazole compound include the following compounds, which represent Compound 1, Compound 2, Compound 3, Compound 4, and Compound 5:

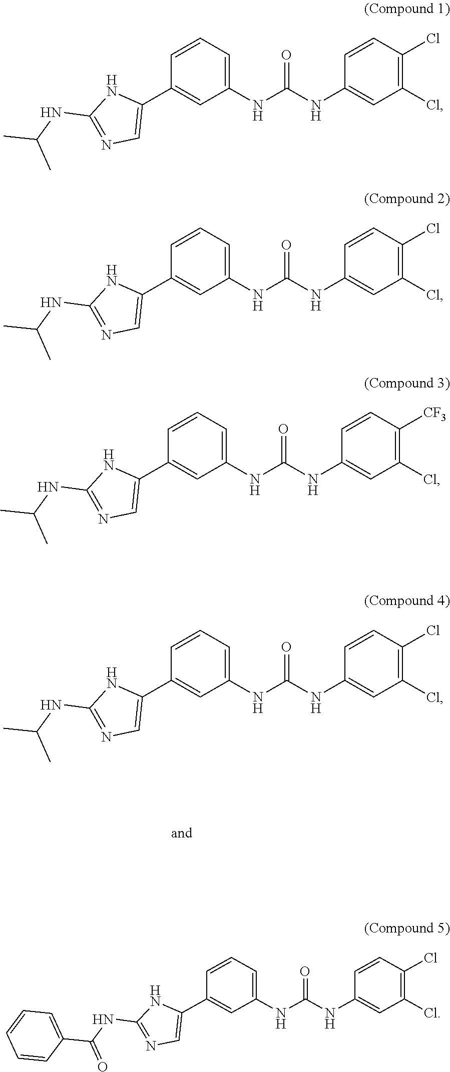 [SMILES: CC(C)Nc1ncc(-c2cccc(NC(=O)Nc3ccc(Cl)c(Cl)c3)c2)[nH]1 ; CC(C)Nc1ncc(-c2cccc(NC(=O)Nc3ccc(Cl)c(Cl)c3)c2)[nH]1 ; CC(C)Nc1ncc(-c2cccc(NC(=O)Nc3ccc(C(F)(F)F)c(Cl)c3)c2)[nH]1 ; CC(C)Nc1ncc(-c2cccc(NC(=O)Nc3ccc(Cl)c(Cl)c3)c2)[nH]1 ; O=C(Nc1cccc(-c2cnc(NC(=O)c3ccccc3)[nH]2)c1)Nc1ccc(Cl)c(Cl)c1]

The 2-(substituted-amino)-imidazole compounds may be used in a method of inhibiting, reducing growth of or destroying gram positive bacteria. The method includes contacting the gram-positive bacteria with an effective amount of the 2-(substituted-amino)-imidazole compound of the general formula (Formula I), a salt, enantiomer or derivative thereof, and with an additional antibacterial compound separately, simultaneously, or sequentially:

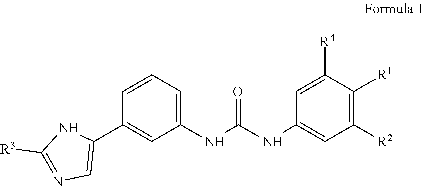

wherein,

R¹, R², and R⁴, which may be the same or different, are each selected from the group consisting of hydrogen, lower alkyl, halogen, and haloalkyl, and

R³ is lower alkylamino, lower isoalkylamino or benzamide.

In embodiments, the gram-positive bacteria may be contacted with one or more of Compounds 1-5 in combination with an additional antibacterial compound. When the 2-(substituted-amino)-imidazole compound and the additional antibacterial compound are applied in combination, they provide an antibiotic potentiation effect against the gram-positive bacteria. In embodiments, the additional antibacterial compound comprises penicillin, daptomycin, vancomycin, oxacillin, linezolid, or related antibiotic(s).

The 2-(substituted-amino)-imidazole compounds may be used in a method of enhancing antibacterial activity of a first antibacterial compound against gram positive bacteria. The method includes contacting the gram-positive bacteria with an effective amount the first antibacterial compound and an effective amount of a 2-(substituted-amino)-imidazole antibacterial compound, a salt, enantiomer or derivative thereof, separately, simultaneously, or sequentially. In embodiments, the 2-(substituted-amino)-imidazole antibacterial compound is one or more of Compounds 1-5. In embodiments, the first antibacterial compound comprises penicillin, daptomycin, vancomycin, oxacillin, linezolid, or related antibiotic(s). The gram-positive bacteria may comprise one or more of the genera Actinomyces, Bacillus, Listeria, Lactococcus, Staphylococcus, Streptococcus, Enterococcus, Mycobacterium, Corynebacterium, or Clostridium.

The 2-(substituted-amino)-imidazole antibacterial compounds are effective in reducing the minimum inhibitory concentration (MIC) of a first antibacterial compound against gram-positive bacteria. In fact, embodiments of the 2-(substituted-amino)-imidazole antibacterial compound can reduce the MIC of a first antibacterial compound by at least 10×. That is, in a direct comparison, the MIC of a first antibacterial compound used in combination with a 2-(substituted-amino)-imidazole antibacterial compound can be 10 times less than the MIC of the first antibacterial compound used alone against the same gram-positive bacteria. In fact, embodiments of the 2-(substituted-amino)-imidazole antibacterial compound can reduce the MIC of a first antibacterial compound by at least 15×, at least 25×, and by at least 30×. Varying amounts and concentrations of 2-(substituted-amino)-imidazole antibacterial compound are effective and may be used. For example, concentrations in a range of 0.1 to 100 μM may be suitable. Exemplary concentrations include 1 μM to 5 μM, for example, 2 μM to 4 μM.

The 2-(substituted-amino)-imidazole compounds may be used in a method of treating a subject suffering from an infection contributed to or caused by gram-positive bacteria. The method includes administering an effective amount of a therapeutically active 2-(substituted-amino)-imidazole compound, an enantiomer or salt thereof, and an additional antibacterial compound, separately, simultaneously, or sequentially. The gram-positive bacteria may comprise one or more of the genera Actinomyces, Bacillus, Listeria, Lactococcus, Staphylococcus, Streptococcus, Enterococcus, Mycobacterium, Corynebacterium, or Clostridium. For example, the gram-positive bacteria may comprise one or more of the genera Staphylococcus. In embodiments, the 2-(substituted-amino)-imidazole antibacterial compound is one or more of Compounds 1-5. In embodiments, the first antibacterial compound comprises penicillin, daptomycin, vancomycin, oxacillin, linezolid, or related antibiotic(s).

The subject may be human or animal. The therapeutically active 2-(substituted-amino)-imidazole compound and the antibacterial compound may be administered orally, topically to the site of infection, intravenously, transmucosally, or transdermally. In embodiments, the infection may be infections affecting animals, including, for example, bovine mastitis.

Therapeutic or pharmaceutical compositions may comprise a 2-(substituted-amino)-imidazole compound and an additional antibiotic combined with a variety of carriers to treat illnesses caused by gram-positive bacteria. The carrier may suitably contain minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts. Glycerin or glycerol (1,2,3-propanetriol) is commercially available for pharmaceutical use. DMSO is an aprotic solvent with an ability to enhance penetration of many locally applied drugs. The carrier vehicle may also include Ringer's solution, a buffered solution, and dextrose solution, particularly when an intravenous solution is prepared.

The effective dosage rates or amounts of a 2-(substituted-amino)-imidazole compound will depend in part on whether the compound is intended to be used therapeutically or prophylactically, the duration of exposure of the subject to the infectious bacteria, the size and weight of the subject, etc. The duration for use of a composition containing the 2-(substituted-amino)-imidazole compound also depends on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day. Any dosage form employed should provide for a minimum number of units for a minimum amount of time. Carriers that are classified as “long” or “slow” release carriers (such as, for example, certain nasal sprays or lozenges) could possess or provide a lower concentration of active units per ml, but over a longer period of time, whereas a “short” or “fast” release carrier (such as, for example, a gargle) could possess or provide a high concentration of active units per ml, but over a shorter period of time. The amount of active units per ml and the duration of time of exposure depend on the nature of infection, whether treatment is to be prophylactic or therapeutic, and other variables. There are situations where it may be necessary to have a much higher unit/ml dosage or a lower unit/ml dosage.

A mild surfactant can be included in a therapeutic or pharmaceutical composition in an amount effective to potentiate the therapeutic effect of the 2-(substituted-amino)-imidazole compound. Suitable mild surfactants include, inter alia, esters of polyoxyethylene sorbitan and fatty acids (Tween series), octylphenoxy polyethoxy ethanol (Triton-X series), n-Octyl-.beta.-D-glucopyranoside, n-Octyl-.beta.-D-thioglucopyranoside, n-Decyl-.beta.-D-glucopyranoside, n-Dodecyl-.beta.-D-glucopyranoside, and biologically occurring surfactants, e.g., fatty acids, glycerides, monoglycerides, deoxycholate and esters of deoxycholate.

Preservatives may also be used in a therapeutic or pharmaceutical composition. The use of preservatives can aid in preventing or diminishing microorganism growth if the composition is microbially contaminated. Suitable preservatives may include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof.

Modes of application of the therapeutic composition comprising 2-(substituted-amino)-imidazole compounds and antibiotic(s) include, but are not limited to direct, indirect, carrier and special means or any combination of means. Direct application of the composition may be by any suitable means to directly bring the 2-(substituted-amino)-imidazole compound and antibiotic in contact with the site of infection or bacterial colonization, such as to dermal or skin applications (for example topical ointments or formulations), etc.

Additionally, the mode of application for 2-(substituted-amino)-imidazole compounds and antibiotic(s) can include a number of different types and combinations of carriers which include, but are not limited to an aqueous liquid, an alcohol base liquid, a water soluble gel, a lotion, an ointment, a nonaqueous liquid base, a mineral oil base, a blend of mineral oil and petrolatum, lanolin, liposomes, protein carriers such as serum albumin or gelatin, powdered cellulose carmel, and combinations thereof. The carriers of topical compositions may comprise semi-solid and gel-like vehicles that include a polymer thickener, water, preservatives, active surfactants or emulsifiers, antioxidants, and a solvent or mixed solvent system.

A 2-(substituted-amino)-imidazole compound and antibiotic(s) may be administered for use by any pharmaceutically applicable or acceptable means including topically, orally or parenterally. For example, the 2-(substituted-amino)-imidazole compounds and antibiotic(s) can be administered intramuscularly, intrathecally, subdermally, subcutaneously, or intravenously to treat infections by gram-positive bacteria. In cases where parenteral injection is the chosen mode of administration, an isotonic formulation can be used. Suitable additives for isotonicity can include sodium chloride, dextrose, mannitol, sorbitol and lactose. In some cases, isotonic solutions such as phosphate buffered saline may be preferred. Stabilizers include gelatin and albumin. A vasoconstriction agent can be added to the formulation. The pharmaceutical preparations can be provided sterile and pyrogen free.

Compositions comprising a 2-(substituted-amino)-imidazole compound and antibiotic(s) may be administered orally, topically to the site of an infection, transmucosally, transdermally or intravenously. Accordingly, compositions comprising a 2-(substituted-amino)-imidazole compound and antibiotic(s) may be formulated as sterile pharmaceutical compositions comprising a pharmaceutically acceptable carrier or excipient. Such carriers or excipients are well known to one of skill in the art and may include, for example, water, saline, phosphate buffered saline, dextrose, glycerol, ethanol, ion exchangers, alumina, aluminium stearate, lecithin, serum proteins, such as serum albumin, buffer substances such as phosphates, glycine, sorbic acid, potassium sorbate, partial glyceride mixtures of saturated vegetable fatty acids, lactic acid, water salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cyclodextrins, such as α-cyclodextrin, β-cyclodextrin, sulfobutylether₇-β-cyclodextrin and hydroxypropyl-β-cyclodextrin, cellulose-based substances, polyethylene glycol, sodium carboxymethylcellulose, polyacrylates, waxes, polyethylene-polypropylene-block polymers, polyethylene glycol and wool fat and the like, and combinations thereof.

For a 2-(substituted-amino)-imidazole compound, a therapeutically effective dose can be estimated initially either in cell culture assays or in animal models, for example, mice, rabbits, dogs, or pigs. The animal model can also be used to achieve a desirable concentration range and route of administration. Such information can then be used to determine useful doses and routes for administration in humans or other animals. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

The effective dosage rates or amounts of the 2-(substituted-amino)-imidazole compounds and antibiotic(s) to be administered, and the duration of treatment will depend in part on the seriousness of the infection, the weight of the patient, particularly human, the duration of exposure of the subject to the infectious bacteria, the number of square centimeters of skin or tissue that are infected, the depth of the infection, the seriousness of the infection, and a variety of a number of other variables. A composition comprising a 2-(substituted-amino)-imidazole compound and antibiotic(s) may be topically applied from once to several times a day or a week, and may be applied for a short, such as days or up to several weeks, or long term period, such as many weeks or up to months. The usage may last for days or weeks. Any dosage form employed should provide for a minimum number of units for a minimum amount of time. The concentration of the 2-(substituted-amino)-imidazole compound and antibiotic(s) believed to provide for a therapeutically effective amount or dosage may be selected as appropriate.

The 2-(substituted-amino)-imidazole compounds and antibiotic(s) of use and application in the compositions and methods described herein may be administered simultaneously or subsequently. The 2-(substituted-amino)-imidazole compounds and antibiotic(s) may be administered in a single dose or multiple doses, singly or in combination. The 2-(substituted-amino)-imidazole compounds and antibiotic(s) may be administered by the same mode of administration or by different modes of administration, and may be administered once, twice or multiple times, one or more in combination or individually. Thus, a 2-(substituted-amino)-imidazole compound may be administered in an initial dose followed by a subsequent dose or doses, particularly depending on the response and bacterial killing or decolonization, and may be combined or alternated with antibiotic dose(s).

The term “Therapeutically effective amount” means that amount of a drug, compound, antimicrobial, antibody, polypeptide, or pharmaceutical agent that will elicit the biological or medical response of a subject that is being sought by a medical doctor or other clinician. In particular, with regard to gram-positive bacterial infections and growth of gram-positive bacteria, the term “effective amount” is intended to include an effective amount of a compound or agent that will bring about a biologically meaningful decrease in the amount of or extent of infection of gram-positive bacteria, including having a bacteriocidal and/or bacteriostatic effect.

The term “treating” or “treatment” of any disease or infection refers, in one embodiment, to reducing the disease or infection (i.e., arresting the disease or growth of the infectious agent or bacteria or reducing the manifestation, extent or severity of at least one of the clinical symptoms thereof). In yet another embodiment, “treating” or “treatment” refers to modulating the disease or infection, either physically, (e.g., stabilization of a discernible symptom), physiologically, (e.g., stabilization of a physical parameter), or both. In a further embodiment, “treating” or “treatment” relates to slowing the progression of a disease or reducing an infection.

It is noted that in the context of treatment methods which are carried out in vivo or medical and clinical treatment methods in accordance with the present application and claims, the term subject is intended to refer to a human or an animal.

The terms “gram-positive bacteria”, “Gram-positive bacteria”, “gram-positive” and any variants not specifically listed, may be used herein interchangeably, and as used throughout the present application and claims refer to Gram-positive bacteria which are known and/or can be identified by the presence of certain cell wall and/or cell membrane characteristics and/or by staining with Gram stain. Gram positive bacteria are known and can readily be identified and may be selected from but are not limited to the genera Listeria, Staphylococcus, Streptococcus, Enterococcus, Mycobacterium, Corynebacterium, and Clostridium, and include any and all recognized or unrecognized species or strains thereof.

The phrase “pharmaceutically acceptable” refers to molecular entities and compositions that are physiologically tolerable and do not typically produce an allergic or similar untoward reaction, such as gastric upset, dizziness and the like, when administered to a human.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Synthetic Preparations

2-(substituted-amino)-imidazole compounds containing an aliphatic substituent on N-2 of the 2-aminoimidazole ring were prepared by the synthetic scheme shown below.

The desired aliphatic substituent was established in the first step by displacement of the chlorine atom of 2-chloropyrimidine. The resulting product was condensed with 2′-bromo-3-nitroacetophenone providing an imidazopyrimidine salt which was isolated by filtration. Treatment of the salt with hydrazine monohydrate resulted in a Dimroth rearrangement that provided the core 4-phenylimidazole with aminoaliphatic substitution in the 2-position. Protection of an internal nitrogen of the imidazole ring was accomplished by treatment with di-tert-butyldicarbonate. Reduction of the nitro group under catalytic hydrogenation conditions provided the aniline, which is condensed with an isocyanate to provide the penultimate compound. Deprotection of the tert-butoxycarbonyl group with an appropriate acid provided the desired final compound as a salt.

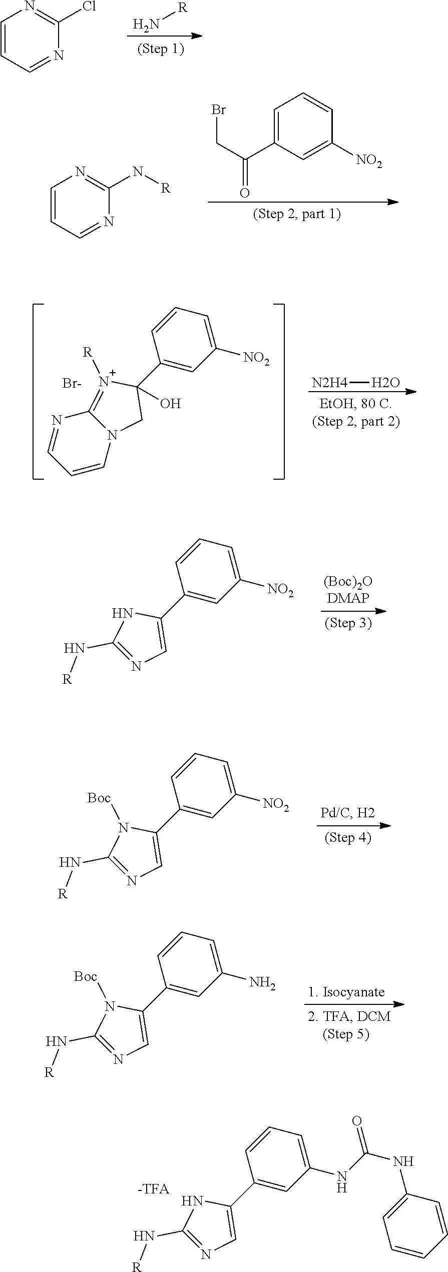

The 2-(substituted-amino)-imidazole compounds with an N-acyl substituent in the 2-position of the imidazole ring was prepared by the synthetic scheme shown below.

Condensation of 2′-bromo-3-nitroacetophenone with tert-butoxycarbonyl guanidine provided the 2-amino-4-nitrophenylimidazole product with one of the internal imidazole nitrogens protected by a tert-butoxycarbonyl group. Amide formation was accomplished by reacting the previous product with the desired carboxylic acid in the presence of a suitable coupling reagent. Reduction of the nitro group under catalytic hydrogenation conditions resulted in the corresponding aniline which was then condensed with the desired isocyanate to provide the final urea.

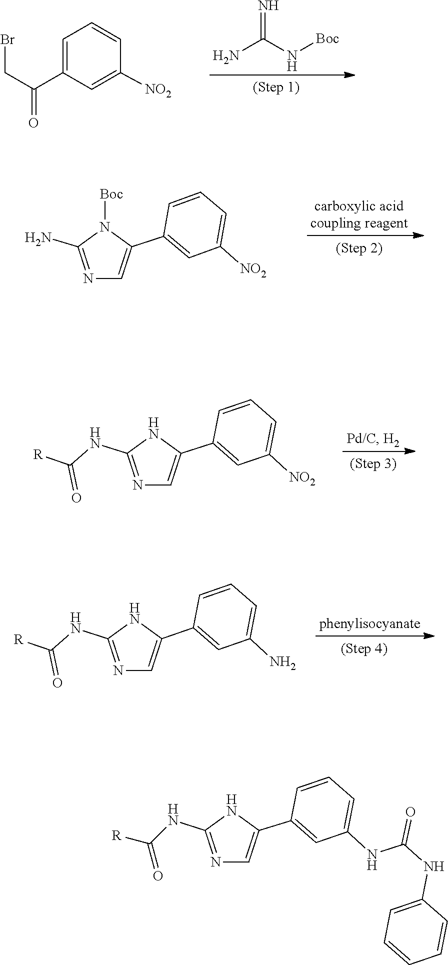

### Example 2

### Synthesis of Intermediate A: tert-butyl 5-(3-aminophenyl)-2-(isopropylamino)-1H-imidazole-1-carboxylate

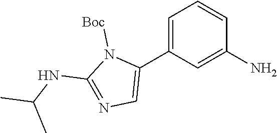 [SMILES: CC(C)Nc1ncc(-c2cccc(N)c2)n1C(=O)OC(C)(C)C]

### Step 1: Synthesis of 2-N-isopropylpyrimidine

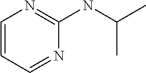

In a sealable flask in ethanol was dissolved in 2-chloropyrimidine (1 eq) and isopropylamine (2.5 eq). The reaction was heated to 85° C. then sealed and stirred overnight. The resulting reaction was cooled to room temperature and the ethanol was removed by rotary evaporation. The residue was taken up in acetonitrile and the resulting mixture was filtered to remove solids. The filtrate was used as is in the next step.

### Step 2: N-isopropyl-5-(3-nitrophenyl)-1H-imidazol-2-amine

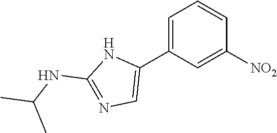 [SMILES: CC(C)Nc1ncc(-c2cccc([N+](=O)[O-])c2)[nH]1]

Part 1: The filtrate from the previous step was treated with 2′-bromo-3-nitroacetophenone (1 eq), and the resulting mixture was heated to 85° C. then sealed. The reaction was stirred at 85° C. for three days then allowed to cool to room temperature. The resulting solids were collected on filter and washed with acetonitrile.
Part 2: The washed solids were taken up in EtOH and treated with an excess of hydrazine monohydrate. The yellow solution was heated to a mild reflux for one hour then the solvents were removed by rotary evaporation. The residue was dissolved in water and extracted with ethyl acetate. The organic extracts were dried over sodium sulfate; the drying salts were removed by filtration, and the filtrate was concentrated to a dark red oil which was used as is in the next step.

### Step 3: tert-Butyl 2-(isopropylamino)-5-(3-nitrophenyl)-1H-imidazole-1-carboxylate

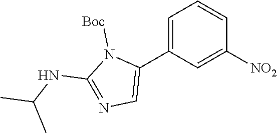 [SMILES: CC(C)Nc1ncc(-c2cccc([N+](=O)[O-])c2)n1C(=O)OC(C)(C)C]

The product from the previous step was dissolved in THF and treated with di-tert-butyldicarbonate (1.5 eq) and a catalytic amount of N′,N′-dimethyl-4-aminopyridine. The reaction was stirred at room temperature for 2 hr then concentrated to dryness. The residue was purified by silica gel chromatography to provide the title compound.

### Step 4: tert-butyl 5-(3-aminophenyl)-2-(isopropylamino)-1H-imidazole-1-carboxylate

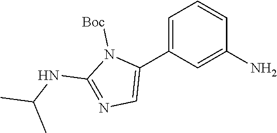 [SMILES: CC(C)Nc1ncc(-c2cccc(N)c2)n1C(=O)OC(C)(C)C]

The product from Step 3 (1 mass) was dissolved in 1:1 ethyl acetate/methanol and placed under a nitrogen atmosphere. To this was added 10% Pd/C (0.1 mass) and the nitrogen atmosphere was replaced with a hydrogen atmosphere. The reaction was stirred at room temperature until the starting material was consumed (about 2 hr). The reaction was then filtered through Celite. The filtrate was concentrated to dryness and purified by silica gel chromatography.

### EXAMPLE

### Synthesis of Intermediate B: tert-butyl-5-(3-aminophenyl)-2-(methylamino)-1H-imidazole-1-carboxylate

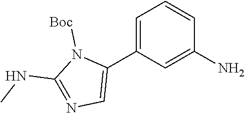 [SMILES: CNc1ncc(-c2cccc(N)c2)n1C(=O)OC(C)(C)C]

### Step 1: Synthesis of 2-N-methylpyrimidine

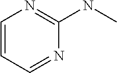

Dissolved 2-chloropyrimidine in IPA, and added methylamine (30% in EtOH) and potassium carbonate (2 eq). Heated the reaction in a microwave at 150° C. for 1 hr. Concentrated to remove solvent and took up residue in water. Extracted with ethyl acetate then dried the extracts over sodium sulfate. Removed the drying salts by filtration and concentrated the filtrate to an oil.

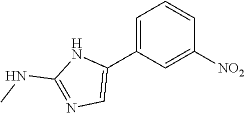 [SMILES: CNc1ncc(-c2cccc([N+](=O)[O-])c2)[nH]1]

Part 1: Dissolved the crude product from Step 1 in acetonitrile. Added 2′-bromo-3-nitroacetophenone (1.1 eq) and heated in a microwave at 120° C. for 1 hour. Collected the resulting solids on filter. Washed the solids with acetonitrile and air-dried to obtain an off-white powder which was used in the next step.
Part 2: Dissolved the washed solids from the previous step in EtOH and added hydrazine hydrate (12 eq). Heated at 80° C. for 1 hr. Concentrated to dryness and took up residue in a dilute sodium bicarbonate solution. Extracted with ethyl acetate and dried the extracts over sodium sulfate. Removed the drying salts by filtration and concentrated the filtrate to dryness. Took up the residue in ethyl acetate and sonicated. Collected the resulting solids on filter to obtain the title product.

### Step 3: tert-Butyl 2-(methylamino)-5-(3-nitrophenyl)-1H-imidazole-1-carboxylate

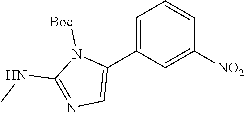 [SMILES: CNc1ncc(-c2cccc([N+](=O)[O-])c2)n1C(=O)OC(C)(C)C]

The product from the previous step was dissolved in THF and treated with di-tert-butyldicarbonate (1.5 eq) and a catalytic amount of N′,N′-dimethyl-4-aminopyridine. The reaction was stirred at room temperature for 2 hr then concentrated to dryness. The residue was purified by silica gel chromatography to provide the title compound.

### Step 4: tert-butyl 5-(3-aminophenyl)-2-(isopropylamino)-1H-imidazole-1-carboxylate

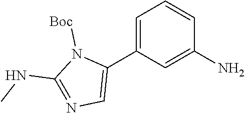 [SMILES: CNc1ncc(-c2cccc(N)c2)n1C(=O)OC(C)(C)C]

Dissolved starting material in methanol and placed the reaction under a nitrogen atmosphere. Added 10% Pd/C (10 mass %) and replaced the nitrogen atmosphere with a hydrogen atmosphere. Stirred at room temperature for 30 min. Diluted with ethyl acetate and filtered through Celite. Concentrated the filtrate to an oil and purified by silica gel chromatography. Collected the desired fractions and concentrated to a light yellow oil to obtain Key Intermediate B.

### Example 4

### Synthesis of Intermediate C: N-(5-(3-aminophenyl)-1H-imidazol-2-yl)benzamide

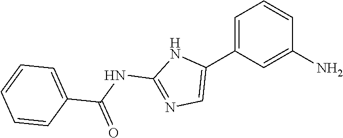 [SMILES: Nc1cccc(-c2cnc(NC(=O)c3ccccc3)[nH]2)c1]

### Step 1: tert-butyl 2-amino-5-(3-nitrophenyl)-1H-imidazole-1-carboxylate

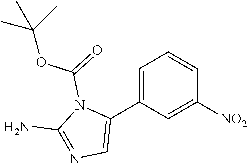 [SMILES: CC(C)(C)OC(=O)n1c(-c2cccc([N+](=O)[O-])c2)cnc1N]

Dissolved 2′-bromo-3-nitroacetophenone (1 eq) in tetrahydrofuran and added tert-butoxycarbonylguanidine (1.2 eq) and a catalytic amount of N′,N′-dimethyl-4-aminopyridine. Heated the reaction at 70° C. for several hours. Concentrated the reaction to dryness and triturated the residue in diethyl ether. Collected the resulting solids on filter to obtain the desired product.

### Step 2: N-(5-(3-nitrophenyl)-1H-imidazol-2-yl)benzamide

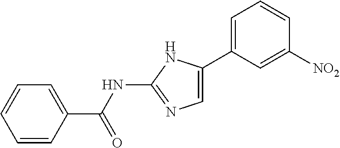 [SMILES: O=C(Nc1ncc(-c2cccc([N+](=O)[O-])c2)[nH]1)c1ccccc1]

The product from the previous step (1 eq) was dissolved in DMF using external heat, then was added diisopropylethylamine (4 eq) and benzoic acid (1.4 eq). Added solid HATU (1.2 eq) and stirred at 65° C. for 2 days. Concentrated to remove DMF, took up residue in a saturated solution of sodium bicarbonate and extracted with ethyl acetate. The organic extracts were dried over sodium sulfate then the drying salts were removed by filtration and the filtrate was concentrated to dryness. The residue was purified by silica gel chromatography to obtain the desired product which no longer contained the tert-butoxycarbonyl protecting group.

### Step 3: N-(5-(3-aminophenyl)-1H-imidazol-2-yl)benzamide

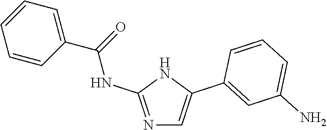 [SMILES: Nc1cccc(-c2cnc(NC(=O)c3ccccc3)[nH]2)c1]

The starting material obtained from the previous step was dissolved in EtOAc/MeOH (4:1) and placed under a nitrogen atmosphere. To this was added 10% Pd/C (30 mass %) and the reaction was placed under a hydrogen atmosphere. Stirred at room temperature for 2 hr then removed the palladium catalyst by filtration through Celite. Concentrated the filtrate to dryness and triturated the residue in diethyl ether. Collected the resulting yellow solids on filter to obtain Intermediate C.

### Example 5

### General Procedure for the Preparation of 1-(3-(2-(alkylamino)-1H-imidazol-5-yl)phenyl)-3-phenylureas

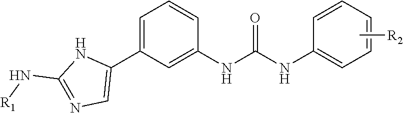

### From an Intermediate C and a Phenylisocyanate:

Dissolved the Intermediate (1 eq) in THF and added a THF solution of the phenylisocyanate (1.2 eq). Stirred at room temperature until the Intermediate had been consumed. Concentrated the reaction to remove THF and took up the residue in DCM. Added an equal volume of TFA and stirred at room temperature for 3 hr. Concentrated to dryness and reconcentrated from DCM/MeOH. Triturated the residue in an appropriate solvent and collected the solids on filter to obtain the desired product as a TFA salt. If trituration did not produce a solid, then the residue was purified by silica gel chromatography using a mixture of DCM/MeOH/NH3 to elute the product, providing the desired material as a free base.

The following compounds were prepared from Intermediate A and the appropriate isocyanate:

### 1. 1-(3,4-dichlorophenyl)-3-(3-(2-(Isopropylamino)-1H-Imidazol-5-yl)phenyl)urea

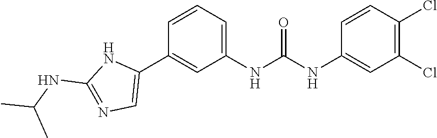 [SMILES: CC(C)Nc1ncc(-c2cccc(NC(=O)Nc3ccc(Cl)c(Cl)c3)c2)[nH]1]

Compound 1: TFA salt: ¹H NMR (d6-DMSO): 12.22 ppm (br s, 2H), 9.32 ppm (s, 1H), 9.13 ppm (s, 1H), 7.93 ppm (d, J-9 Hz, 1H), 7.87 ppm (d, J-3 Hz, 1H), 7.76 ppm (s, 1H), 7.46 ppm (d, J-9 Hz, 1H), 7.35-7.16 ppm (m, 5H), 3.86-3.67 ppm (m, 1H), 1.15 ppm (d, J-6 Hz, 6H)

### 2. 1-(3,5-dichlorophenyl)-3-(3-(2-(Isopropylamino)-1H-Imidazol-5-yl)phenyl)urea

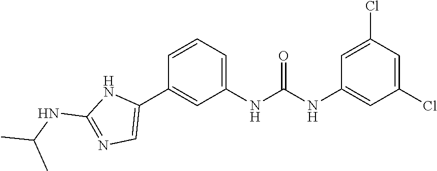 [SMILES: CC(C)Nc1ncc(-c2cccc(NC(=O)Nc3cc(Cl)cc(Cl)c3)c2)[nH]1]

Compound 2: TFA salt: ¹H NMR (d6-DMSO): 12.22 ppm (br s, 2H), 9.38 ppm (s, 1H), 9.20 ppm (s, 1H), 7.90 ppm (d, J-6 Hz, 1H), 7.76 ppm (t, J-3 Hz, 1H), 7.50 ppm (d, J-3 Hz, 2H), 7.37-7.16 ppm (m, 4H), 7.10 ppm (t, J-3 Hz, 1H), 3.84-3.68 ppm (m, 1H), 1.16 ppm (d, J-6 Hz, 6H)

### 3. 1-(3-chloro-4-(trifluoromethyl)phenyl)-3-(3-(2-(Isopropylamino)-1H-imidazol-5-yl)phenyl)urea

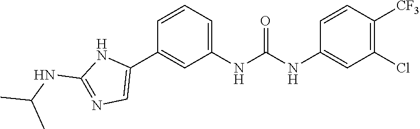 [SMILES: CC(C)Nc1ncc(-c2cccc(NC(=O)Nc3ccc(C(F)(F)F)c(Cl)c3)c2)[nH]1]

Compound 3: TFA salt: ¹H NMR (d6-DMSO): 12.22 ppm (br s, 2H), 9.67 ppm (s, 1H), 9.28 ppm (s, 1H), 7.91 ppm (m, 2H), 7.77 ppm (d, J-3 Hz, 1H), 7.76 ppm (s, 1H), 7.68 ppm (d, J-9 Hz, 1H), 7.42 ppm (d, J-9 Hz, 1H), 7.35-7.20 ppm (m, 4H), 3.82-3.68 ppm (m, 1H), 1.18 ppm (d, J-6 Hz, 6H)

The following compound was prepared from Intermediate B and 3,4-dichlorophenylisocyanate:

### 4. 1-(3,4-dichlorophenyl)-3-(3-(2-(methylamino)-1H-Imidazol-5-yl)phenyl)urea

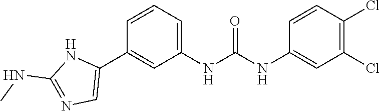 [SMILES: CNc1ncc(-c2cccc(NC(=O)Nc3ccc(Cl)c(Cl)c3)c2)[nH]1]

Compound 4: TFA salt: ¹H NMR (d6-DMSO): 12.37 ppm (br s, 2H), 9.45 ppm (s, 1H), 9.23 ppm (s, 1H), 7.85 ppm (s, 1H), 7.76 ppm (br s, 1H), 7.44 ppm (d, J-6 Hz, 1H), 7.36-7.15 ppm (m, 4H), 7.05 ppm (s, 1H), 6.87 ppm (s, 1H), 2.84 ppm (d, J-6 Hz, 3H)

The following compound was prepared from Intermediate C and 3,4-dichlorophenylisocyanate:

### 5. N-(5-(3-(3-(3,4-dichlorophenyl)ureido)phenyl)-1H-imidazol-2-yl)benzamide

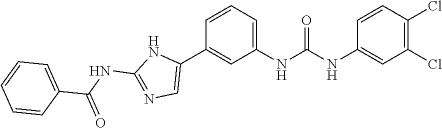 [SMILES: O=C(Nc1cccc(-c2cnc(NC(=O)c3ccccc3)[nH]2)c1)Nc1ccc(Cl)c(Cl)c1]

Compound 5: Free base: ¹H NMR (d6-DMSO): 11.90 ppm (br s, 1H), 11.61 ppm (br s, 1H), 8.98 ppm (s, 1H), 8.75 ppm (s, 1H), 8.01 ppm (d, J-6 Hz, 2H), 7.94 ppm (s, 1H), 7.84 ppm (t, J-3 Hz, 1H), 7.59-7.39 ppm (m, 4H), 7.36-7.03 ppm (m, 5H)

### Example 6

### Assessment of Minimum Inhibitory Concentration

Exemplary embodiments of a 2-(substituted-amino)-imidazole compound in combination with an antibiotic compound were tested to evaluate the effect on minimum inhibitory concentration (MIC).

The minimum inhibitory concentration (MIC) of oxacillin and penicillin were assessed in the presence or absence of a 2-(substituted-amino)-imidazole compound against exemplary bacterial strains, which are shown in Table 1 below. In these experiments, a frozen stock of the bacterial strain was streaked onto a sterile brain heart infusion agar plate and incubated at 37° C. for 16-20 hours. After the incubation period, three to five well-isolated bacterial colonies of the same morphologic type were taken from the brain heart infusion agar plate culture and used to inoculate, using a sterile loop, a tube containing 4 mL to 5 mL of cation adjusted Mueller Hinton (MHII) broth. The culture was allowed to grow in a shaking incubator at 220 rpm at 37° C. until the culture reached logarithmic phase, as determined by the measuring of and achieving an optical density (OD) of 0.3 to 0.5 measured at 600 nm. The bacterial concentration of the culture was then adjusted with the addition of MHII broth to achieve the equivalent of 1×10⁶ colony-forming unit (CFU)/mL using the approximation that 1×10⁹ CFU/mL is equal to OD600 nm=1. Cells were then treated with the designated amount of test compound (shown in Table 1) or a solvent control (dimethyl sulfoxide) for 30 minutes at ambient conditions. Cells were then transferred into 96-well round bottom plates and exposed to 2-fold serial dilutions of the denoted antibiotic. Plates were incubated at 37° C. for 16 to 20 hours; at which time, the lowest concentration of antibiotic that resulted in no visible bacteria pellet formation (i.e., prevention of bacteria pellet formation) was recorded as the MIC value. The MIC values between test compound-treated samples versus solvent-treated control samples were compared and listed in the table below to assess the antibiotic potentiation activity of each test compound.

Table 1 shows the minimum inhibitory concentration of penicillin against various Staphylococcus aureus, and Coagulase-negative staphylococci strains in the presence and absence of the 2-(substituted-amino)-imidazole compound of Compound 1. The concentration of each compound used in μM is shown in parenthesis.

TABLE 1

| | Minimum Inhibitory Concentration of | |
|---|---|---|
| | penicillin in μg/mL | |
| | No imidazole | |
| Strain | compound | +Compound 1 (μM) |
| Staphylococcus aureus | 128 | 8 |
| Xen30 | | (4) |
| Staphylococcus aureus | 64 | 2 |
| B8 | | (2) |
| Staphylococcus aureus | 16 | 1 |
| B12 | | (2) |
| Coagulase-negative | 32 | 0.125 |
| staphylococci CN58 | | (2) |
| Coagulase-negative | 2 | 0.125 |
| staphylococci CN8 | | (2) |
| Coagulase-negative | 8 | 0.5 |
| staphylococci CN74 | | (2) |

Table 2 shows the minimum inhibitory concentration of Oxacillin against Staphylococcus aureus in the presence and absence of Compounds 1, 2, 3, 4, and 5. The concentration (μM) of the compound used is given in parentheses.

TABLE 2

| | Minimum Inhibitory Concentration of Oxacillin in μg/mL | | | | | |
|---|---|---|---|---|---|---|
| | No imidazole | + Compound | + Compound | + Compound | + Compound | + Compound |
| Strain | compound | 1 (μM) | 2 (μM) | 3 (μM) | 4 (μM) | 5 (μM) |
| Staphylococcus | 128 | 4 | 2 | 8 | 16 | 1 |
| aureus Xen30 | | (1) | (4) | (7) | (2) | (7) |

1. A therapeutically active 2-(substituted-amino)-imidazole compound of the general formula (Formula I), a salt, enantiomer or derivative thereof: 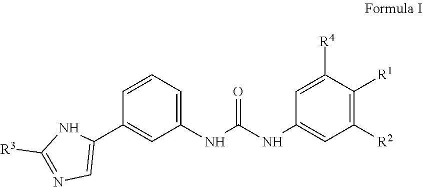
wherein,
R¹, R² and R⁴, which may be the same or different, are each selected from the group consisting of hydrogen, lower alkyl, halogen, and haloalkyl, and
R³ is lower alkylamino, lower isoalkylamino or benzamide.

2. The compound of claim 1, wherein the compound is selected from the group consisting of the following compounds:

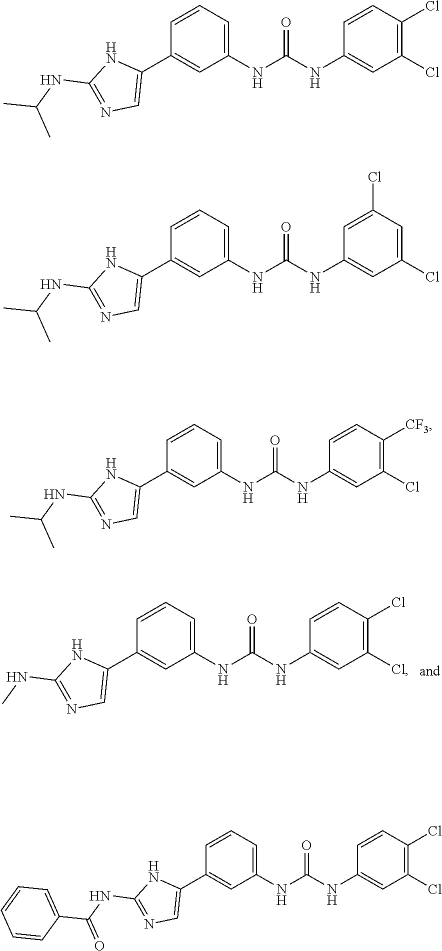 [SMILES: CC(C)Nc1ncc(-c2cccc(NC(=O)Nc3ccc(Cl)c(Cl)c3)c2)[nH]1 ; CC(C)Nc1ncc(-c2cccc(NC(=O)Nc3cc(Cl)cc(Cl)c3)c2)[nH]1 ; CC(C)Nc1ncc(-c2cccc(NC(=O)Nc3ccc(C(F)(F)F)c(Cl)c3)c2)[nH]1 ; CNc1ncc(-c2cccc(NC(=O)Nc3ccc(Cl)c(Cl)c3)c2)[nH]1 ; O=C(Nc1cccc(-c2cnc(NC(=O)c3ccccc3)[nH]2)c1)Nc1ccc(Cl)c(Cl)c1]

3. A method of inhibiting, reducing growth of or destroying gram positive bacteria comprising contacting the gram positive bacteria with an effective amount of a 2-(substituted-amino)-imidazole compound of the general formula (Formula I), a salt, enantiomer or derivative thereof, and with an additional antibacterial compound separately, simultaneously, or sequentially: 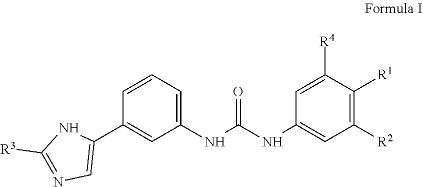
wherein,
R¹, R², and R⁴, which may be the same or different, are each selected from the group consisting of hydrogen, lower alkyl, halogen, and haloalkyl, and
R³ is lower alkylamino, lower isoalkylamino or benzamide.

4. The method of claim 3, wherein the imidazole compound is selected from the group consisting of the following compounds:

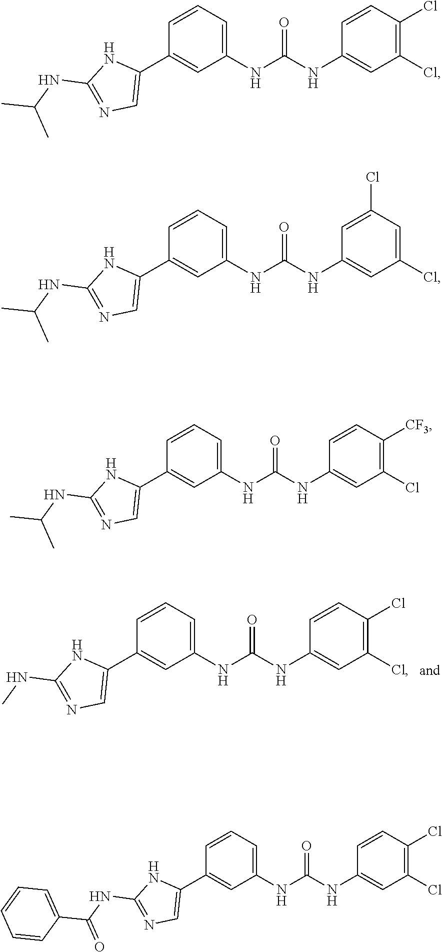 [SMILES: CC(C)Nc1ncc(-c2cccc(NC(=O)Nc3ccc(Cl)c(Cl)c3)c2)[nH]1 ; CC(C)Nc1ncc(-c2cccc(NC(=O)Nc3cc(Cl)cc(Cl)c3)c2)[nH]1 ; CC(C)Nc1ncc(-c2cccc(NC(=O)Nc3ccc(C(F)(F)F)c(Cl)c3)c2)[nH]1 ; CNc1ncc(-c2cccc(NC(=O)Nc3ccc(Cl)c(Cl)c3)c2)[nH]1 ; O=C(Nc1cccc(-c2cnc(NC(=O)c3ccccc3)[nH]2)c1)Nc1ccc(Cl)c(Cl)c1]

5. The method of claim 3, wherein the compound of the general formula (Formula I), a salt, enantiomer or derivative thereof and the additional antibacterial compound provide an antibiotic potentiation effect against the gram-positive bacteria.

6. The method of claim 3, wherein the additional antibacterial compound comprises penicillin, daptomycin, vancomycin, oxacillin, linezolid, or related antibiotic(s).

7. A method of enhancing antibacterial activity of a first antibacterial compound against gram positive bacteria, comprising contacting the gram positive bacteria with an effective amount the first antibacterial compound and an effective amount of a 2-(substituted-amino)-imidazole antibacterial compound, a salt, enantiomer or derivative thereof, separately, simultaneously, or sequentially.

8. The method of claim 7, wherein the 2-(substituted-amino)-imidazole antibacterial compound is selected from the group consisting of the following compounds:

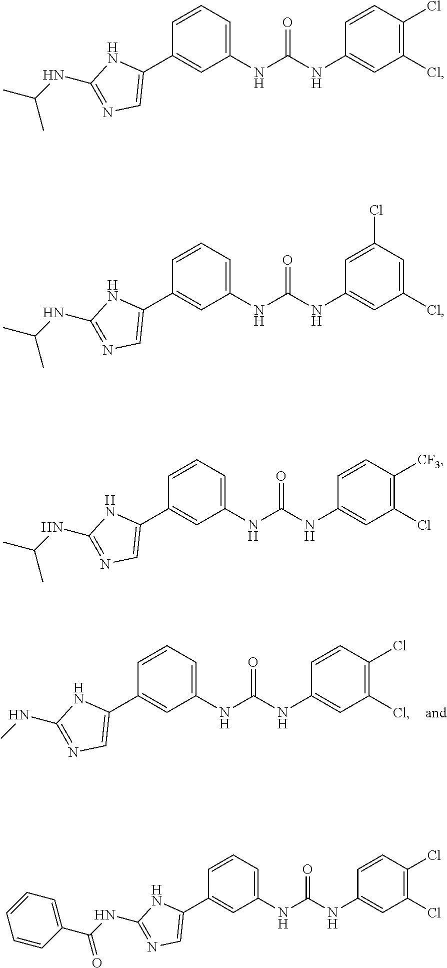 [SMILES: CC(C)Nc1ncc(-c2cccc(NC(=O)Nc3ccc(Cl)c(Cl)c3)c2)[nH]1 ; CC(C)Nc1ncc(-c2cccc(NC(=O)Nc3cc(Cl)cc(Cl)c3)c2)[nH]1 ; CC(C)Nc1ncc(-c2cccc(NC(=O)Nc3ccc(C(F)(F)F)c(Cl)c3)c2)[nH]1 ; CNc1ncc(-c2cccc(NC(=O)Nc3ccc(Cl)c(Cl)c3)c2)[nH]1 ; O=C(Nc1cccc(-c2cnc(NC(=O)c3ccccc3)[nH]2)c1)Nc1ccc(Cl)c(Cl)c1]

9. The method of claim 7, wherein the first antibacterial compound comprises penicillin, daptomycin, vancomycin, oxacillin, linezolid, or related antibiotic(s).

10. The method of claim 3, wherein the gram positive bacteria comprise one or more of the genera Actinomyces, Bacillus, Listeria, Lactococcus, Staphylococcus, Streptococcus, Enterococcus, Mycobacterium, Corynebacterium, or Clostridium.

11. The method of claim 7, wherein the minimum inhibitory concentration (MIC) of the first antibacterial compound is reduced by at least 10× when the first antibacterial compound is applied in combination with the 2-(substituted-amino)-imidazole antibacterial compound, a salt, enantiomer or derivative thereof, wherein applied in combination includes application to the gram positive bacteria separately, simultaneously, or sequentially.

12. The method of claim 11, wherein the MIC of the first antibacterial compound is reduced by at least 15×.

13. The method of claim 12, wherein the MIC of the first antibacterial compound is reduced by at least 25×.

14. Method of treating a subject suffering from an infection contributed to or caused by gram-positive bacteria, comprising administering an effective amount of a therapeutically active 2-(substituted-amino)-imidazole compound, an enantiomer or salt thereof, and an additional antibacterial compound, separately, simultaneously, or sequentially.

15. The method of claim 14, wherein the gram-positive bacteria comprise one or more of the genera Actinomyces, Bacillus, Listeria, Lactococcus, Staphylococcus, Streptococcus, Enterococcus, Mycobacterium, Corynebacterium, or Clostridium.

16. The method of claim 14, wherein the gram-positive bacteria comprise one or more of the genera Staphylococcus.

17. The method of claim 14, wherein the therapeutically active 2-(substituted-amino)-imidazole compound is selected from the group consisting of the following compounds:

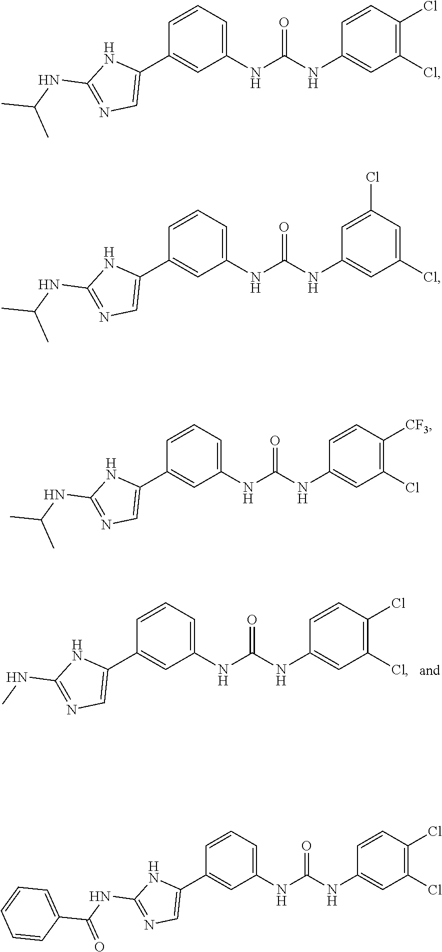 [SMILES: CC(C)Nc1ncc(-c2cccc(NC(=O)Nc3ccc(Cl)c(Cl)c3)c2)[nH]1 ; CC(C)Nc1ncc(-c2cccc(NC(=O)Nc3cc(Cl)cc(Cl)c3)c2)[nH]1 ; CC(C)Nc1ncc(-c2cccc(NC(=O)Nc3ccc(C(F)(F)F)c(Cl)c3)c2)[nH]1 ; CNc1ncc(-c2cccc(NC(=O)Nc3ccc(Cl)c(Cl)c3)c2)[nH]1 ; O=C(Nc1cccc(-c2cnc(NC(=O)c3ccccc3)[nH]2)c1)Nc1ccc(Cl)c(Cl)c1]

18. The method of claim 14, wherein the first antibacterial compound comprises penicillin, daptomycin, vancomycin, oxacillin, linezolid, or related antibiotic(s).

19. (canceled)

20. The method of claim 14, wherein the therapeutically active 2-(substituted-amino)-imidazole compound and the antibacterial compound are administered orally, topically to the site of infection, intravenously, transmucosally, or transdermally.

21. The method of claim 14, wherein the infection is bovine mastitis
